(19) United States
(12) Patent Application Publication
(10) Pub. No.: US 2023/0253077 A1
(43) Pub. Date: Aug. 10, 2023

(54) ANIMAL CARE SUPPORT SYSTEM, ANIMAL CARE SUPPORT SERVER, AND ANIMAL CARE SUPPORT PROGRAM

(71) Applicant: PECO INC., Tokyo (JP)

(72) Inventor: Jun OKAZAKI, Tokyo (JP)

(21) Appl. No.: 17/907,644

(22) PCT Filed: Mar. 31, 2020
(86) PCT No.: PCT/JP2020/014787

§ 371 Date: Apr. 3, 2023

(51) Int. Cl. G16H 10/20; A01K 29/00; G06V 40/10

(52) U.S. Cl. CPC G16H 10/20; A01K 29/005; G06V 40/10

(57) ABSTRACT

A medical institution to easily conduct medical examination regarding an animal. An animal medical care support system that supports medical care of an animal, the system comprising: a medical inquiry storage unit that stores question items in association with animal species; an animal species specifying unit that specifies the animal species of the animal scheduled to receive medical examination at a veterinary medical institution; a question transmission unit that reads out the question items corresponding to the specified animal species and transmits them to a portable terminal operated by a breeder of the animal; an answer reception unit that receives answers to the question items from the portable terminal; and an answer output unit that outputs the answer to a medical terminal of a medical personnel of the veterinary medical institution.

### TECHNICAL FIELD

The present invention relates to an animal medical care support system, an animal medical care support server and an animal medical care support program.

### BACKGROUND ART

A system for specifying diseases by medical examination of an animal has been proposed (see Patent Literature 1).

### PRIOR ART LIST

### Patent Literature

Patent Literature 1: Japanese Unexamined Patent Publication No. 2002-132918

### SUMMARY OF THE INVENTION

### Technical Problem

However, in the system described in Patent Literature 1, it is assumed that the answer to a medical inquiry is used for automatic disease determination, and when a patient visits a medical institution, it is necessary to ask medical questions again.

The present invention has been made in view of such a background, and an object of the present invention is to provide a technology that makes it easy for medical institutions to perform medical inquires related to an animal.

### Technical Solution

The main invention of the present invention for achieving the above object is an animal medical care support system that supports medical care of an animal, the system comprising: a medical inquiry storage unit that stores question items in association with animal species; an animal species specifying unit that specify the animal species of the animal that is scheduled to visit the medical/veterinary institution; a question transmission unit that reads out the question items corresponding to the specified animal species and transmits them to a portable terminal operated by a breeder of the animal; an answer reception unit that receives answers to the question items from the portable terminal; and an answer output unit that outputs the answer to a medical terminal of a medical personnel of the veterinary medical institution.

### Advantageous Effects

According to the present invention, a medical institution can easily conduct medical examination related to an animal.

### BRIEF DESCRIPTION OF THE DRAWING

FIG. 1 is a diagram showing the overall configuration example of an animal medical care support system according to the present embodiment;

FIG. 2 is a diagram showing a hardware configuration example of a portable terminal 1;

FIG. 3 is a diagram showing a software configuration example of the portable terminal 1;

FIG. 4 is a diagram showing a hardware configuration example of a medical institution terminal 2;

FIG. 5 is a diagram showing a software configuration example of the medical institution terminal 2;

FIG. 6 is a diagram showing a hardware configuration example of a management server 3;

FIG. 7 is a diagram showing a software configuration example of the management server 3;

FIG. 8 is a diagram for explaining the flow of medical examination registration processing;

FIG. 9 is a diagram showing the flow of processing related to medical examination before an animal receives a medical examination at a veterinary medical institution;

FIG. 10 is a diagram for explaining the flow of the determination processing of the medical institution;

FIG. 11 is a diagram showing an example of a screen 50 displayed on the portable terminal 1 for a medical examination in a chat format;

FIG. 12 is an example of another medical examination screen that is not in a chat format;

FIG. 13 is an example of a medical examination screen that is not in a chat format;

FIG. 14 is an example of another medical examination screen that is not in a chat format; and

FIG. 15 is an example of another medical examination screen that is not in a chat format.

### DETAILED DESCRIPTION OF THE EMBODIMENTS

### Summary of Invention

The contents of the embodiment of the present invention will be listed and described. The present invention has the following configuration.

### Item 1

An animal medical care support system that supports medical care of an animal, the system comprising:

a medical inquiry storage unit that stores question items in association with animal species;

an animal species specifying unit that specifies the animal species of the animal scheduled to receive medical examination at a veterinary medical institution;

a question transmission unit that reads out the question items corresponding to the specified animal species and transmits them to a portable terminal operated by a breeder of the animal;

an answer reception unit that receives answers to the question items from the portable terminal; and

an answer output unit that outputs the answer to a medical terminal of a medical personnel of the veterinary medical institution.

### Item 2

The animal medical care support system according to Item 1 wherein:

the question transmission unit further transmits other question items associated with the answer, in response to the answer from the breeder concerning the question items.

### Item 3

The animal medical care support system according to Items 1 or 2,

which further comprises a hospital visit storage unit that stores the visit history of the animal,

wherein the question transmission unit transmits at least the question items relating to the health check-up or preventive vaccination according to the hospital visit history.

### Item 4

The animal medical care support system according to Item 1 wherein:

the question transmission unit reads out the question items one by one from the medical inquiry storage unit and transmits them to the portable terminal; and

the answer reception unit receives the answer to the question items each time the question items is transmitted.

### Item 5

The animal medical care support system according to Item 1, further comprising:

a message input unit for receiving a message to the breeder from the concerned person; and

a message transmission unit that transmits the received message to the portable terminal.

### Item 6

The animal medical care support system according to Item 1, comprising:

an image reception unit that receives an image of the animal photographed by the portable terminal,

wherein the answer output unit outputs the image together with the answer.

### Item 7

The animal medical care support system according to Item 4 wherein:

the animal species specifying unit specifies the animal species by analysing the image.

### Item 8

The animal medical care support system according to Item 1, comprising:

a question storage unit that stores the questions in association with the animal species and physical feature related to the body of the animal, and

a physical feature specifying unit that specifies the physical feature of the animal,

wherein the question transmission unit reads out the question corresponding to the specified animal species and the physical feature.

### Item 9

The animal medical care support system according to Item 6 wherein:

the physical feature specifying unit specifies the physical feature by analysing the image.

### Item 10

The animal medical care support system according to Item 1, comprising:

a medical examination affirmative/negative response unit that responds whether or not to receive medical examination for the animal at the medical institution.

### Item 11

The animal medical care support system according to Item 8, comprising:

another medical institution specifying unit that specifies another medical institution capable of medical examination of the animal when the medical examination is impossible, wherein:

the medical examination affirmative/negative response unit transmits information related to the other medical institution to the portable terminal.

### Item 12

The animal medical care support system according to one of Items 1 to 11 wherein:

the response reception unit receives information input by the breeder to the portable terminal, and also includes an image display unit that displays the image information on a doctor terminal operated by a veterinarian at the medical institution when the animal receives a medical examination at the medical institution.

### Item 13

The animal medical care support system according to one of Items 1 to 12 wherein:

the portable terminal prompts the breeder to input at least one of text information, image information, or voice information in response to the question items, and

the answer output unit outputs at least one of the text information, the image information, or the voice information input by the breeder to the medical terminal.

### Item 14

An animal medical care support server that supports medical care of an animal, the server comprising:

a medical inquiry storage unit that stores question items in association with animal species;

an animal species specifying unit that specifies the animal species of the animal scheduled to receive medical examination at a veterinary medical institution;

a question transmission unit that reads out the question items corresponding to the specified animal species and transmits them to a portable terminal operated by a breeder of the animal;

an answer reception unit that receives answers to the questions from the portable terminal; and

an answer output unit that outputs the answer to a medical terminal of a medical personnel of the veterinary medical institution.

### Item 15

An animal medical care support program that supports medical care of an animal, the system, the program making an animal medical care support server function as:

a medical inquiry storage unit that stores question items in association with animal species;

an animal species specifying unit that specifies the animal species of the animal scheduled to receive medical examination at a veterinary medical institution;

a question transmission unit that reads out the question items corresponding to the specified animal species and transmits them to a portable terminal operated by a breeder of the animal;

an answer reception unit that receives answers to the question items from the portable terminal; and

an answer output unit that outputs the answer to a medical terminal of a medical personnel of the veterinary medical institution.

### System Overview

An animal medical care support system according to an embodiment of the present invention will be described below. The animal medical care support system of the present embodiment performs a medical examination related to the health condition of an animal to an animal caregiver (an animal owner, a family member living with the owner, etc.) prior to medical care of the animal. In the animal medical care support system of the present embodiment, medical examinations are conducted in a chat format. In addition, the answers to the medical examinations are transmitted to the veterinary medical institution. Since the portable terminal can be used to inform the veterinary medical institution of the condition immediately before the visit to the hospital through medical examinations in this way, smooth medical care can be performed.

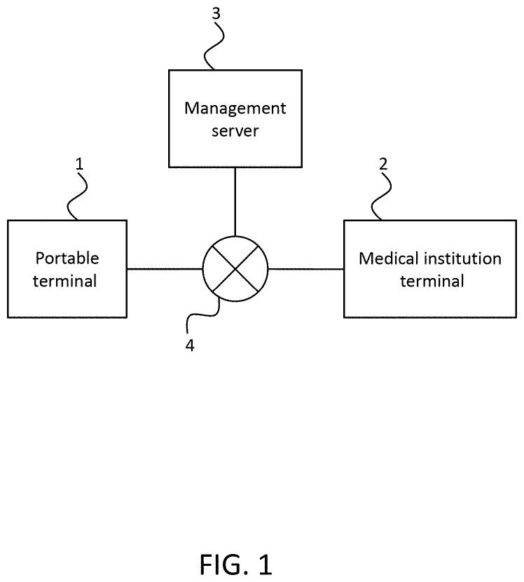

FIG. 1 is a diagram showing the overall configuration example of an animal medical care support system according to the present embodiment. The animal medical care support system of the present embodiment is configured by including a management server 3. The management server 3 is communicably connected to each of a portable terminal 1 and a medical institution terminal 2 via a communication network 4. The communication network 4 is, for example, the Internet, and is composed of a public telephone line network, a mobile telephone line network, a wireless communication path, Ethernet (registered trademark), or the like.

The portable terminal 1 is a portable computer operated by an animal caregiver (owner or person taking care of the animal). The portable terminal 1 is, for example, a smart phone. The portable terminal 1 can be any portable computer such as a tablet computer or a notebook computer.

The medical institution terminal 2 is a computer operated by the veterinary medical personnel (such as nurses and doctors of veterinary hospitals) at a veterinary medical institution. Medical institution terminal 2 is, for example, a tablet computer. The medical institution terminal 2 can be, for example, any computer such as a smart phone or a personal computer.

The management server 3 is a server computer that transmits/receives and records information related to medical examinations. The management server 3 may be, for example, a general-purpose computer such as a workstation or a personal computer or may be a virtual computer that is logically realized by cloud computing.

### Portable Terminal 1

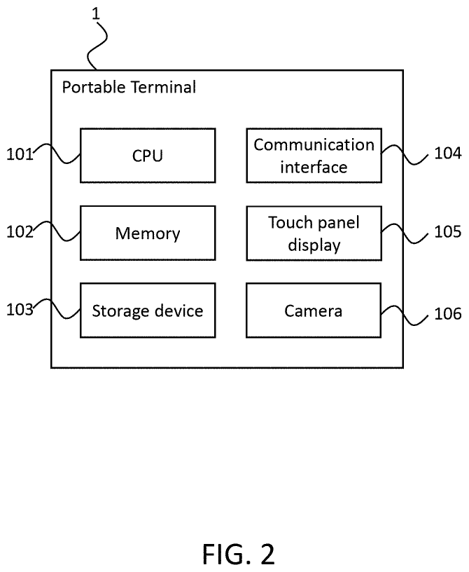

FIG. 2 is a diagram showing a hardware configuration example of the portable terminal 1. The portable terminal 1 includes a CPU 101, a memory 102, a storage device 103, a communication interface 104, a touch panel display 105 and a camera 106. The storage device 103 is, for example, a hard disk drive, solid state drive, flash memory, or the like that stores various data and programs. The communication interface 104 is an interface for connecting to the communication network 4, and examples thereof include an adapter for connecting to Ethernet (registered trademark), a modem for connecting to a public telephone network, a wireless communication device for performing wireless communication, USB (Universal Serial Bus) connector or RS232C connector for serial communication, and the like. The touch panel display 105 is a device that inputs and outputs data. The portable terminal 1 may include, for example, input devices such as a keyboard, a mouse, a button, and a microphone, and output devices such as a display, a printer, and a speaker, instead of the touch panel display 105.

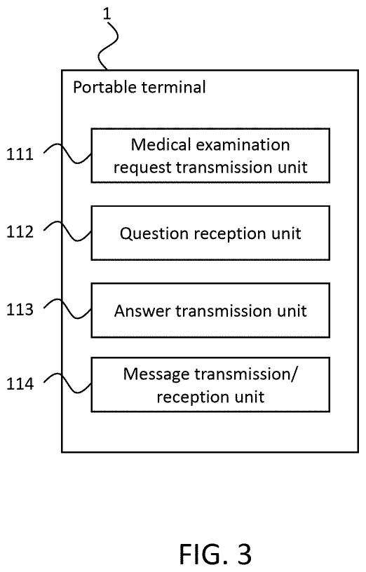

FIG. 3 is a diagram showing a software configuration example of the portable terminal 1. The portable terminal 1 includes a medical examination request transmission unit 111, a question reception unit 112, an answer transmission unit 113 and a message transmission/reception unit 114. Further, each of these functional units is realized by allowing CPU 101 installed in the portable terminal 1 to read out a program stored in the storage device 103 to the memory 102 and execute the program.

The medical examination request transmission unit 111 transmits a request for receiving a medical examination at a veterinary medical institution such as an animal hospital (hereinafter referred to as medical examination request). In the present embodiment, the medical examination request transmission unit 111 transmits a medical examination request to the management server 3. The medical examination request transmission unit 111 sets and transmits an owner ID indicating a caregiver (owner of the animal being cared for) and an animal ID indicating the animal to be examined in a medical examination request. The medical examination request transmission unit 111 may transmit the name of the owner and the name and species of the animal in place of the owner ID and the animal ID, for example, in the case of the first visit or the like. The medical examination request transmission unit 111 may receive designation of a desired veterinary medical institution from a caregiver and may include a medical institution ID indicating the received veterinary medical institution in the medical examination request.

The question reception unit 112 receives a question related to the medical examination, and the answer transmission unit 113 receives and transmits an answer to the question from a caregiver. The question reception unit 112 and the answer transmission unit 113 transmit and receive answers and questions as messages in chat format. In the present embodiment, questions are transmitted from the management server 3. The question reception unit 112 displays the received question on the touch panel display 105, and the answer transmission unit 113 receives an answer to the question from the caregiver and transmits the received answer to the management server 3.

The message transmission/reception unit 114 can exchange various messages with the veterinary medical personnel in a chat format. The message transmission/reception unit 114 can receive and transmit a message input from a caregiver, and also receive and output a message from a veterinary medical personnel member. In the present embodiment, it is assumed that the message transmitted by the message transmission/reception unit 114 is transmitted to the management server 3 and transferred from the management server 3 to the medical institution terminal 2. The message transmission/reception by the message transmission/reception unit 114 is also performed in a chat format, similarly to the question reception unit 112 and the answer transmission unit 113.

### Medical Institution Terminal 2

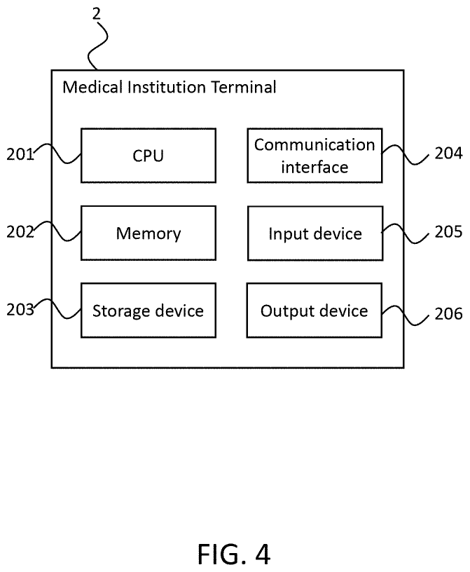

FIG. 4 is a diagram showing a hardware configuration example of the medical institution terminal 2. The medical institution terminal 2 includes a CPU 201, a memory 202, a storage device 203, a communication interface 204, an input device 205 and an output device 206. The storage device 203 is, for example, a hard disk drive, solid state drive, flash memory, etc., which stores various data and programs. The communication interface 204 is an interface for connecting to the communication network 4, and examples thereof include an adapter for connecting to Ethernet (registered trademark), a modem for connecting to a public telephone network, a wireless communication device for wireless communication, a USB (Universal Serial Bus) connector or RS232C connector for serial communication, and the like. The input device 205 is, for example, a keyboard, a mouse, a touch panel, button, a microphone, etc. for inputting data. The output device 206 is, for example, a display, a printer, a speaker, etc. that outputs data.

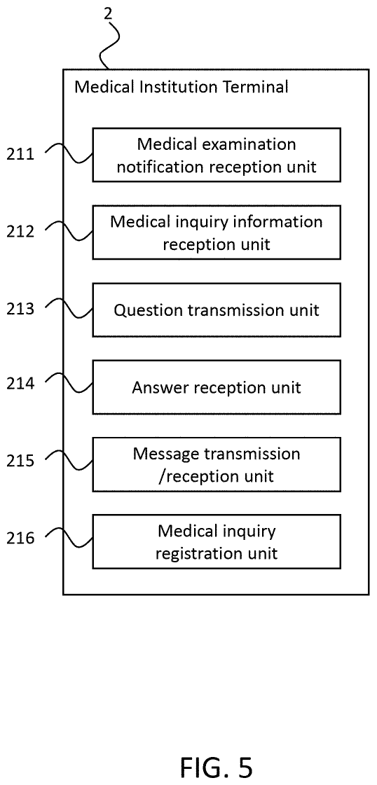

FIG. 5 is a diagram showing a software configuration example of the medical institution terminal 2. The medical institution terminal 2 includes a medical examination notification reception unit 211, a medical inquiry information reception unit 212, a question transmission unit 213, an answer reception unit 214, a message transmission/reception unit 215 and a medical inquiry registration unit 216.

The medical examination notification reception unit 211 receives a notification indicating that there is an animal wishing to receive a medical examination at a veterinary institution (hereinafter referred to as medical examination notification). In the present embodiment, the medical examination notification is transmitted from the management server 3. An animal ID indicating an animal wishing to receive a medical examination and an owner ID indicating the owner of the animal are set in the medical examination notification (if previously registered in the system).

The medical inquiry information reception unit 212 receives information including answers to pre-registered medical inquiries. In the present embodiment, the medical inquiry information is transmitted from the management server 3.

The question transmission unit 213 transmits a question related to the medical examination, and the answer reception unit 214 receives an answer to the question. The question transmission unit 213 and the answer reception unit 214 transmit and receive answers and questions as messages in chat format.

The question transmission unit 213 receives and transmits input of questions that veterinary medical personnel such as doctors and nurses pose to caregivers. In the present embodiment, the question transmission unit 213 transmits the received question to the management server 3, which is transmitted from the management server 3 to the portable terminal 1. If the effect that confirmation from a veterinary institution is necessary is displayed in the medical inquiry information, the question transmission unit 213 can call the veterinary medical personnel's attention (e.g., output of a warning message, warning sound, etc.) to prompt the input of a question.

The answer reception unit 214 receives an answer to a question. In the present embodiment, the caregiver's answer to the question is transmitted from the portable terminal 1 to the management server 3, which is transferred from the management server 3 to the medical institution terminal 2.

The message transmission/reception unit 215 can exchange various messages with the caregiver in a chat format. The message transmission/reception unit 215 can receive and transmit an input of a message from veterinary medical personnel and can receive and output a message from a caregiver. In the present embodiment, it is assumed that the message transmitted by the message transmission/reception unit 215 is also transmitted to the management server 3 and transferred from the management server 3 to the portable terminal 1. It is assumed that message transmission/reception by the message transmission/reception unit 215 is also performed in a chat format, similarly to the question transmission unit 213 and the answer reception unit 214.

The medical inquiry registration unit 216 registers questions regarding medical inquiry in the management server 3 in advance. The medical inquiry registration unit 216 receives input of questions to be asked to the caregiver and conditions (it is possible to set arbitrary conditions, such as what should be asked first, the answer to the question before the question has a specific content, the date falling within a specific period, etc.) for presenting the questions from the veterinary medical personnel. The medical inquiry registration unit 216 can transmit a registration request including a question and its presentation conditions to the management server 3 to register (including update) the medical inquiry.

### Management Server 3

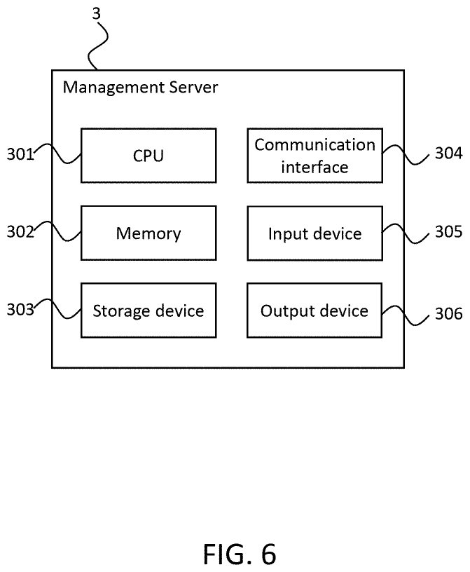

FIG. 6 is a diagram showing a hardware configuration example of the management server 3. The management server 3 includes a CPU 301, a memory 302, a storage device 303, a communication interface 304, an input device 305 and an output device 306. The storage device 303 is, for example, a hard disk drive, solid state drive, or flash memory that stores various data and programs. The communication interface 304 is an interface for connecting to the communication network 4, and examples thereof include an adapter for connecting to Ethernet (registered trademark), a modem for connecting to a public telephone network, a wireless communication device for performing wireless communication, USB (Universal Serial Bus) connector or RS232C connector for serial communication, and the like. The input device 305 is, for example, a keyboard, a mouse, a touch panel, a button, a microphone, and the like for inputting data. The output device 306 is, for example, a display, a printer, a speaker, or the like that outputs data.

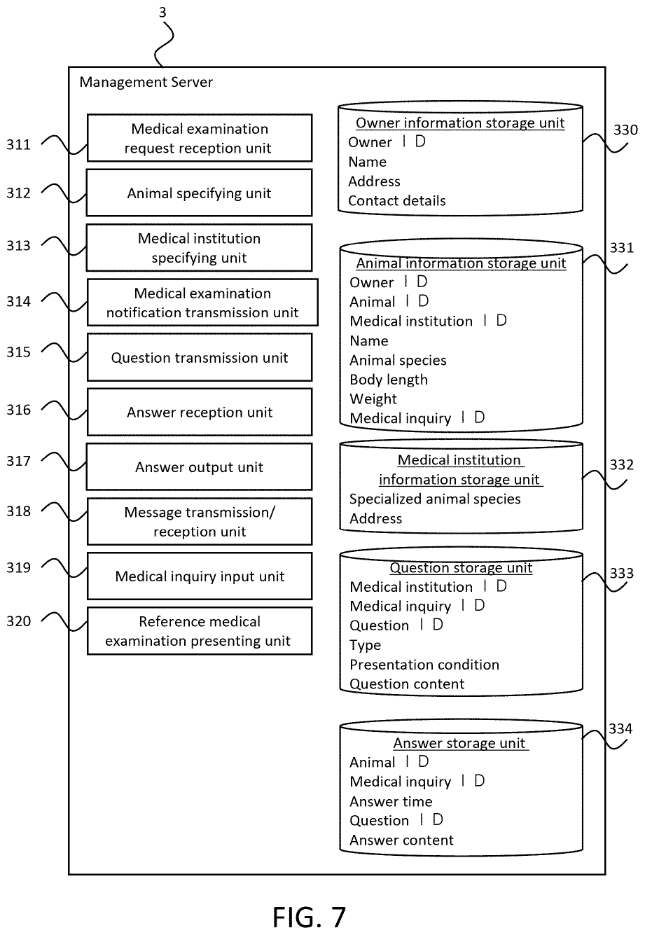

FIG. 7 is a diagram showing a software configuration example of the management server 3. The management server 3 includes each functional unit of a medical examination request reception unit 311, an animal specifying unit 312, a medical institution determination unit 313, a medical examination notification transmission unit 314, a question transmission unit 315, an answer reception unit 316, an answer output unit 317, a message transmission/reception unit 318, a medical inquiry input unit 319, and a reference medical examination presenting unit 320, and each storage unit of an owner information storage unit 330, an animal information storage unit 331, a medical institution information storage unit 332, a question storage unit 333, and an answer storage unit 334.

The owner information storage unit 330 stores information relating to the owner of the animal (hereinafter referred to as owner information). The owner information includes owner ID, name, address, and contact information. The owner ID is information that specifies the owner of the animal. The owner information may include information related to the owner other than the name, address, and contact details.

The animal information storage unit 331 stores information (hereinafter referred to as animal information) related to an animal (animal patient) who receives medical care at a veterinary institution. The animal information includes information such as a medical institution ID indicating an animal patient's family veterinary medical institution, the animal patient's name, animal species, body length, weight, and the like, in association with the owner ID indicating the owner of the animal patient and the animal ID specifying the animal patient. The animal information may also include information related to an animal patient other than these. In addition, in the animal information, a medical inquiry ID can be set to specify a medical inquiry to be performed before medical examination of the animal. The medical inquiry ID can be set by the medical personnel of a veterinary institution after the animal first visits the veterinary institution.

The medical institution information storage unit 332 stores information related to a veterinary medical institution (hereinafter referred to as medical institution information). The medical institution information includes a specialized animal species indicating the animal species that the veterinary institution (where veterinarian affiliated with) specializes in or is good at, and the address of the veterinary institution, in association with the medical institution ID that specifies the veterinary medical institution. The medical institution information may include other information related to the veterinary medical institutions.

The question storage unit 333 stores information including questions related to medical inquires (hereinafter referred to as question information). The question information includes a question ID that specifies a question, a type that indicates the animal species that the question is intended for, conditions for presenting the question, and the content of the question, in association with a medical institution ID that specifies a veterinary medical institution and a medical inquiry ID that indicates a medical inquiry conducted by the veterinary medical institution. The question storage unit 333 can also manage a plurality of medical inquiries in association with, for example, medical institution IDs and types. The content of the question may include answer options. In other words, the content of the question may include a question that is scheduled to be answered in free text, a question that expects an answer that selects one or more from two or three or more options, a question that plans an answer to values (real or integer) within a given range, and the like. In the present embodiment, the answer is to select one of the options. As the presentation condition, arbitrary determinable conditions can be set, such as being the first question to be asked, the answer to a question earlier than the question being specific (e.g., if you answered “no” to the question “do you have an appetite?”, etc.) and the date falling within a specific time period (e.g., when the season at the time of the question is winter, etc.), and the like.

The answer storage unit 334 stores information including answers to questions (hereinafter referred to as answer information). The answer information includes an owner ID indicating the caregiver who responded to the question (the owner of the animal being cared for), an animal ID indicating the animal patient, an inquiry ID indicating the medical inquiry to be answered, the date and time of the response, a question ID indicating the answered question, and the contents of the answer.

The medical examination request reception unit 311 examines a medical examination request from the portable terminal 1. For the medical examination request, processing related to medical examination is performed in response to the reception of the medical examination request.

The animal specifying unit 312 specifies animals related to the medical examination request. The animal specifying unit 312 can retrieve animal information corresponding to the animal ID from the animal information storage unit 331 if an animal ID is included in the medical examination request.

The medical institution determination unit 313 determines the veterinary medical institution to which the animal pertaining to the medical examination request should receive a medical examination. The medical institution determination unit 313 can determine the veterinary medical institution indicated by the medical institution ID as the veterinary medical institution where the animal should receive medical examination, if a medical institution ID is set in the medical examination request. Further, the medical institution determination unit 313 reads out animal information corresponding to the animal ID set in the medical examination request from the animal information storage unit 331, reads out medical institution information corresponding to the animal species indicated by the read animal information from the medical institution information storage unit 332, and may determine, among the read medical institution information, the one whose address is closest to the owner's address (address of owner information corresponding to the owner ID included in the medical examination request) as the veterinary medical institution at which the animal should undergo medical examination.

The medical examination notification transmission unit 314 transmits a notification indicating the effect that the caregiver wishes to have the animal examined at the veterinary medical institution, to the medical institution terminal 2 of the veterinary medical institution determined by the medical institution determination unit 313 (hereinafter referred to as a reception notification.). The medical examinations notification transmission unit 314 sets the animal ID and the owner ID in the medical examination notification and transmits the notification to the medical institution terminal 2, if the animal ID and owner ID are set in the medical examination request.

The question transmission unit 315 transmits a question related to the medical examination. For example, when an animal ID is set in the medical examination request, the question transmission unit 315 reads question information corresponding to the medical inquiry ID of the animal information corresponding to the animal ID from the question storage unit 333 and can transmit to the portable terminal 1 the question information that satisfies the presentation conditions among read-out question information. For example, if the animal ID is not set in the medical examination request, the question transmission unit 315 also reads out question information corresponding to the determined veterinary medical institution and animal species from the question storage unit 333 and can transmit to the portable terminal 1 those pieces of read-out question information that satisfy the presentation conditions.

The answer reception unit 316 receives an answer to questions. The answer reception unit 316 creates reply information including the received answer and registers it in the answer storage unit 334.

The question transmission unit 315 and the answer reception unit 316 transmit and receive questions and answers as messages in a chat format.

The answer output unit 317 transmits answers related to medical inquiries to the medical institution terminal 2. The answer output unit 317 may transmit an answer to the medical institution terminal 2 each time it receives an answer or may collectively transmit the answers to the medical institution terminal 2 after receiving answers to all questions or may be collectively transmitted to the medical institution terminal 2 for each number of answers.

The message transmission/reception unit 318 transmits and receives messages. When any one of the portable terminal 1 or the medical institution terminal 2 receives a message, the message transmission/reception unit 318 transmits the message to the other. Message transmission/reception by the message transmission/reception unit 318 is also performed in a chat format, similarly to the question transmission unit 315 and the answer reception unit 316.

The medical interview input unit 319 accepts input of question information related to the medical interview and registers it in the question storage unit 3333.

The reference medical examination presentation unit 320 can present an example of a question when inputting question information related to medical examination. The reference medical examination presentation unit 320 may present question information as a predetermined default value or may present question information set in another veterinary medical institution as reference information.

### Medical Examination Registration Processing

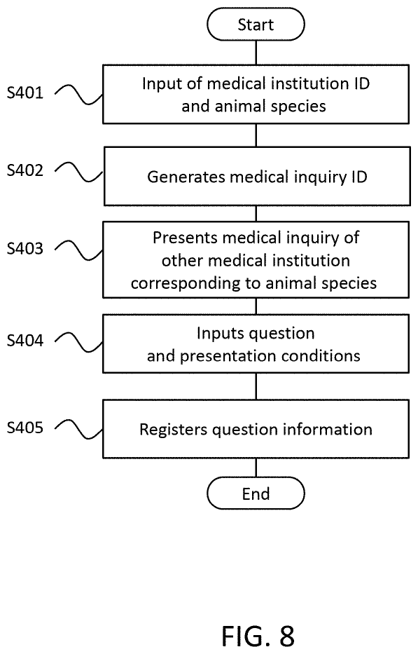

The processing executed in the animal medical care support system of the present embodiment will be described below. FIG. 8 is a diagram for explaining the flow of medical examination registration processing.

The management server 3 receives input of the medical institution ID and the species of the animal to receive a medical examination (S401) and generates a medical examination ID (S402). The management server 3 presents question information of other medical institutions corresponding to the animal species as reference information (S403).

The management server 3 receives input of questions and question presentation conditions from the medical personnel of the veterinary medical institution (S404), and generates question information including the received questions and presentation conditions, medical institution ID, animal species, and medical examination ID, a unique question ID is set and registered in the question storage unit 333 (S405). Further, the steps S404 and S405 receive multiple inputs for multiple questions to be asked in one medical interview.

As described above, question information can be registered for each veterinary institution and animal species.

### Medical Examination Processing

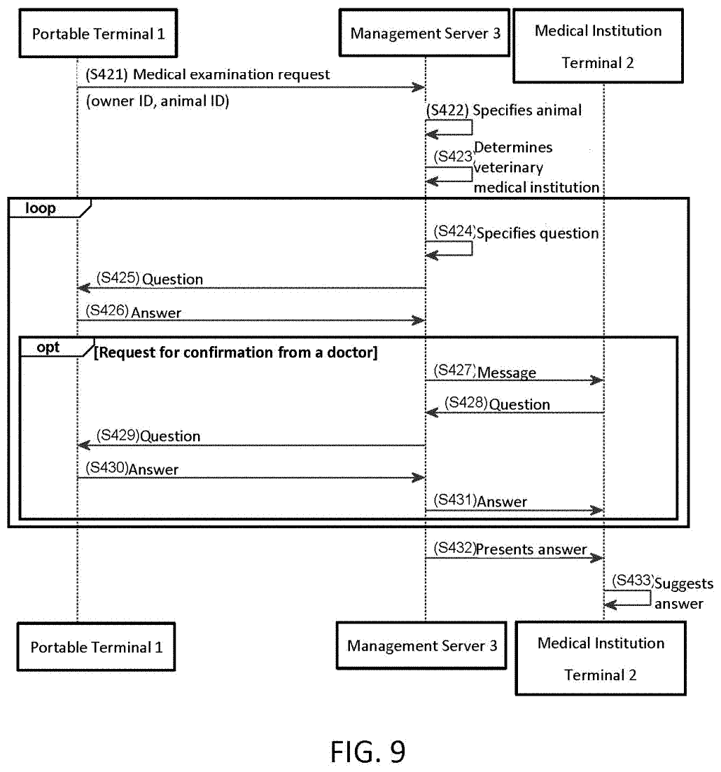

FIG. 9 is a diagram showing the flow of processing related to medical examination before an animal receives a medical examination at a veterinary medical institution.

A medical examination request is transmitted from the mobile terminal 1 to the management server 3 (S421). An owner ID and an animal ID are set in the medical examination request. Further, if there is no medical examination history in the past, the animal species, name, owner's name, and the like are set in the medical examination request.

In response to the medical examination request, the management server 3 specifies the animal wishing to receive a medical examination (S422), and determines the veterinary medical institution (S423).

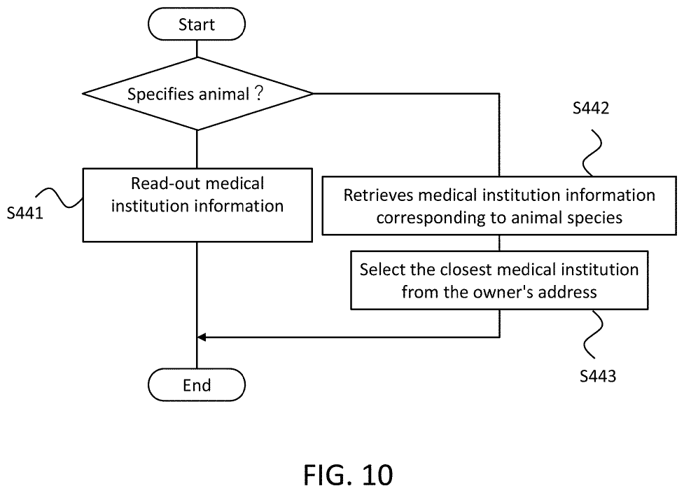

FIG. 10 is a diagram for explaining the flow of the determination processing of the medical institution. If animal information is specified (S441: yes), that is, if an animal ID is set in the medical examination request, the management server 3 reads out the medical institution information corresponding to the medical institution ID set in the animal information from the medical institution information storage unit 332 (S442). Meanwhile, if the animal information is not specified (S441: no), or if the caregiver wishes to receive a medication examination at a veterinary medical institution other than the family veterinary medical institution (the desire to receive a medical examination at a veterinary institution other than the family's veterinary institution can be specified in the consultation request.), the medical institution information corresponding to the animal species set in the medical examination request is retrieved from the medical institution information storage unit 332 (S443), and the one closest to the owner's address which can be set in the medical examination request) among the retrieved medical institution information can be selected (S444).

Returning to FIG. 9, the management server 3 retrieves question information corresponding to the veterinary institution and the animal species from the question storage unit 333, and specifies the question information that satisfies the presentation conditions among the retrieved question information (S424). The management server 3 transmits the question to the portable terminal 1 (S425), and the portable terminal 1 displays the question, and also receives the answer to the question, and transmits it to the management server 3 (S426). Steps S425 and S426 are repeated until there is no next question information (for example, until the question content of the question information becomes information indicating the end of medical inquiry).

Here, depending on the answer from the caregiver, an answer from a veterinarian or a nurse may be necessary. In such a case, a message is transmitted from the management server 3 to the medical institution terminal 2 (S427). Whether or not an answer from a veterinarian or a nurse is required can be set in the question content of the question information. A question and an answer are set in the message. In response to the message, the medical institution terminal 2 receives input of a message such as an additional question from the doctor or the like and transmits it to the management server 3 (S428). The management server 3 transfers the received message such as the additional question or the like to the portable terminal 1 (S429). The portable terminal 1 outputs the message such as the additional question transferred from the management server 3, receives the input of the answer to the message, and transmits it to the management server 3 (S430). The management server 3 transfers the response transmitted from the portable terminal 1 to the medical institution terminal 2 (S431). The exchange of messages related to steps S428 through S431 can also be repeated as many times as the veterinarian needs.

### Screen Example

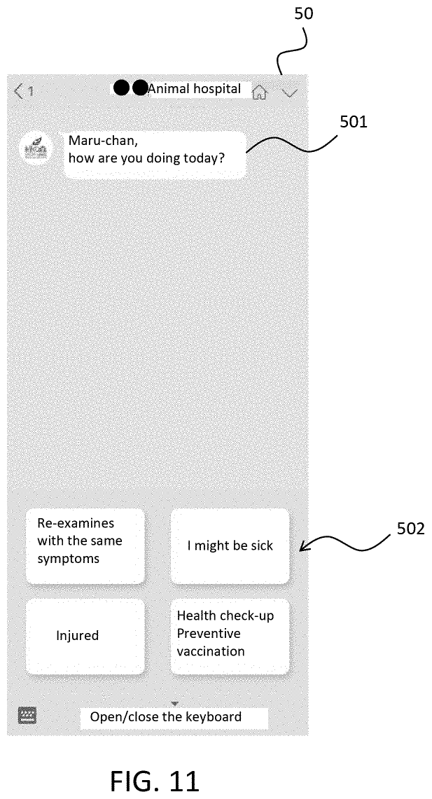

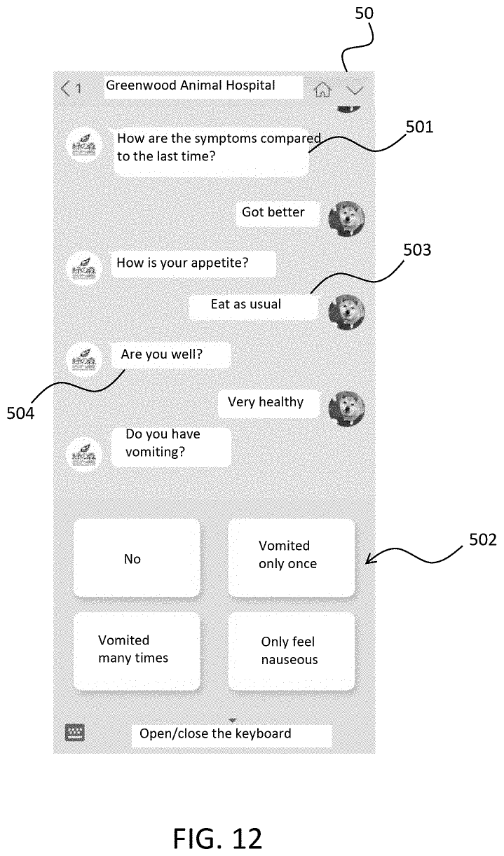

FIG. 11 is a diagram showing an example of a screen 50 displayed on the portable terminal 1 for a medical examination in a chat format. First, a question 501 is automatically output on the screen 50 in the same format as a general chat, which becomes the beginning of the medical inquiry. If the question information related to medical examination includes options, an option 502 is displayed on the screen 50. In addition, if the question information is scheduled to be answered in free text, a text input field can be displayed without displaying the option 502. FIG. 12 is also a diagram showing an example of the screen 50 displayed on the mobile terminal 1 for medical inquiry in chat format. In the present embodiment, the next question 504 in a series of medical inquires is determined according to the answer 503 from the user and the animal species of the animal patient.

After the processing of the question and answer is repeated as described above, the management server 3 transmits an answer to the medical institution terminal 2 transmitted from the portable terminal 1 regarding the medical examination (S432), and the medical institution terminal 2 presents an answer to the medical personnel such as a veterinarian and a nurse or the like (S433).

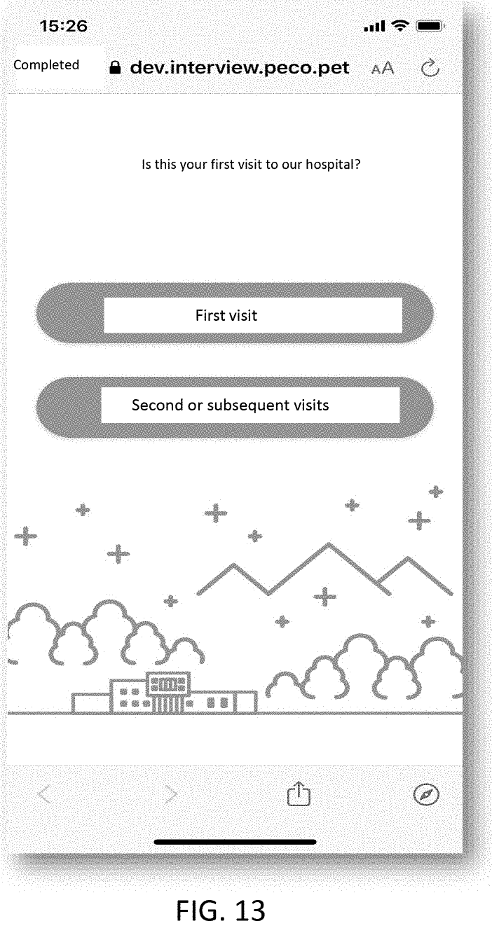

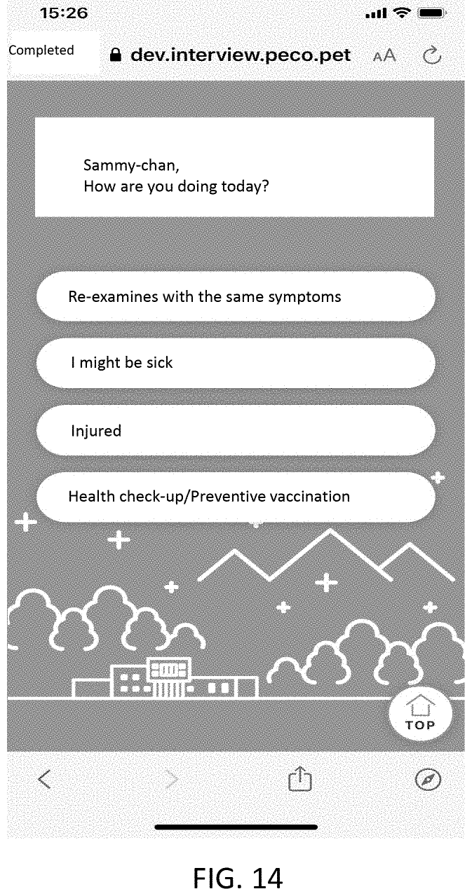

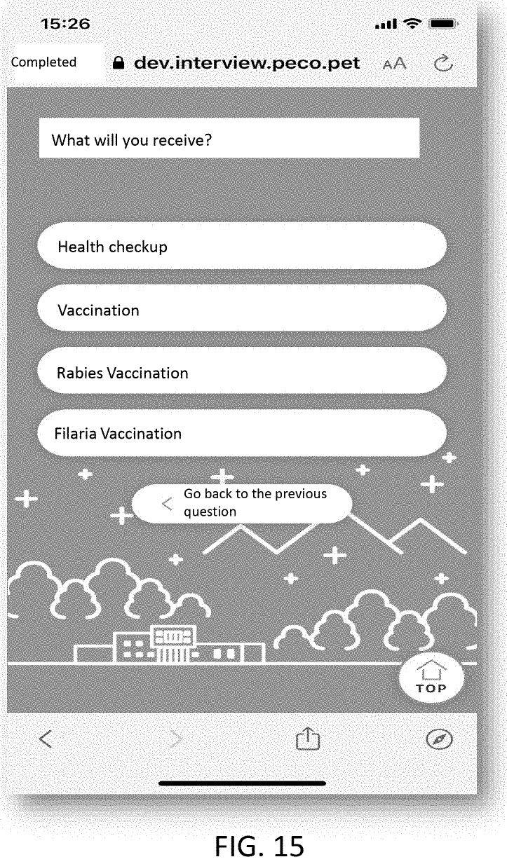

As described above, according to the animal medical care support system of the present embodiment, before a caregiver takes an animal to a veterinary institution, an answer related to medical inquiries is inputted in advance using the portable terminal 1 such as a smart phone, and transmits to the veterinary medical institution. Therefore, veterinary institutions can provide efficient veterinary care. In addition, as shown in FIGS. 13 to 15, the medical examination can be conducted using an interface other than the chat format.

In addition, according to the animal medical care support system of the present embodiment, since it is possible to prepare in advance the optimal medical examination for each animal species, a detailed response becomes possible.

Although the present embodiment has been described above, the embodiment is intended to facilitate understanding of the present invention, and is not intended to interpret as limiting the present invention. The present invention can be modified and improved without departing from its spirit, and the present invention also includes equivalents thereof.

For example, in the present embodiment, the management server 3 is assumed to be one computer, but is not limited thereto and it can be realized by a plurality of computers. For example, a database server may have each storage unit provided in the management server 3, and the management server 3 may access the database server to perform processing. Moreover, it is also possible to disperse and deploy a plurality of functional units included in the management server 3 in a plurality of servers.

Further, in the present embodiment, the medical examination ID is managed for each animal, but is not limited thereto and for example, medical inquiry IDs may be managed for each medical institution ID and animal species, so that medical inquiry can be performed according to the veterinary medical institution and animal species. Further, the same medical inquires can be performed asked regardless of the animal species.

Further, in the present embodiment, question information is registered for each veterinary institution and animal species, but question information can be registered for each animal. In this case, a medical inquiry ID for a specific animal ID can be generated and set in the animal information, and question information set with the inquiry ID can be registered.

In addition, in the present embodiment, it is assumed that the animal ID or animal species is included in the medical examination request, and the animal species is confirmed before the medical examination, but this is not the only option, and medical examination may be conducted in a state where the animal species is unknown. In this case, in the medical examination, questions that can determine the animal species are registered, and the animal species is specified according to the answer to the question. After the animal species is specified, only questions corresponding to the animal species can be transmitted.

### Description of Reference Numerals

1 Mobile terminal
2 Medical institution terminal
3 Management server
111 Medical examination request transmission unit
112 Question reception unit
113 Answer transmission unit
114 Message transmission/reception unit
211 Medical examination notification reception unit
212 Medical inquiry information reception unit
213 Question transmission unit
214 Answer reception unit
215 Message transmission/reception unit
216 Medical inquiry registration unit
311 Medical examination request reception unit
312 Animal specifying unit
313 Medical institution determination unit
314 Medical examination notification transmission unit
315 Question transmission unit
316 Answer reception unit
317 Answer output unit
318 Message transmission/reception unit
319 Medical inquiry input unit
320 Reference medical inquiry presentation unit
330 Owner information storage unit
331 Animal information storage unit
332 Medical institution information storage unit
333 Question storage unit

1. An animal medical care support system that supports medical care of an animal, the system comprising:
a medical inquiry storage unit that stores question items in association with animal species;
an animal species specifying unit that specifies the animal species of the animal scheduled to receive medical examination at a veterinary medical institution;
a question transmission unit that reads out the question items corresponding to the specified animal species and transmits them to a portable terminal operated by a breeder of the animal;
an answer reception unit that receives answers to the question items from the portable terminal; and
an answer output unit that outputs the answer to a medical terminal of a medical personnel of the veterinary medical institution.

2. The animal medical care support system according to claim 1,
wherein the question transmission unit further transmits other question items associated with the answer, in response to the answer from the breeder concerning the question items.

3. The animal medical care support system according to claim 1,
which further comprises a hospital visit storage unit that stores the visit history of the animal,
wherein the question transmission unit transmits at least the question items related to the health check-up or preventive vaccination according to the hospital visit history.

4. The animal medical care support system according to claim 1,
wherein the question transmission unit reads out the question items one by one from the medical inquiry storage unit and transmits them to the portable terminal; and
the answer reception unit receives the answer to the question items each time the question items is transmitted.

5. The animal medical care support system according to claim 1, further comprising:
a message input unit for receiving a message to the breeder from the medical personnel; and
a message transmission unit that transmits the received message to the portable terminal.

6. The animal medical care support system according to claim 1, comprising:
an image reception unit that receives an image of the animal photographed by the portable terminal,
wherein the answer output unit outputs the image together with the answer.

7. The animal medical care support system according to claim 4,
wherein the animal species specifying unit specifies the animal species by analysing the image.

8. The animal medical care support system according to claim 1, comprising:
a question storage unit that stores the questions in association with the animal species and physical feature related to the body of the animal; and
a physical feature specifying unit that specifies the physical feature of the animal,
wherein the question transmission unit reads out the question corresponding to the specified animal species and physical feature.

9. The animal medical care support system according to claim 6, further comprising:
a physical feature specifying unit that specifies a physical feature by analysing the image.

10. The animal medical care support system according to claim 1,
wherein a medical examination affirmative/negative response unit that responds whether or not to receive medical examination for the animal at the veterinary medical institution.

11. The animal medical care support system according to claim 10, comprising:
another veterinary medical institution specifying unit that specifies another veterinary medical institution capable of medical examination of the animal when the medical examination is impossible,
wherein the medical examination affirmative/negative response unit transmits information related to the other veterinary medical institution to the portable terminal.

12. The animal medical care support system according to one of claim 1,
wherein the answer reception unit receives information input by the breeder to the portable terminal, and also includes an image display unit that displays image information on a doctor terminal operated by a veterinarian at the veterinary medical institution when the animal receives a medical examination at the veterinary medical institution.

13. The animal medical care support system according to one of claim 1,
wherein the portable terminal prompts the breeder to input at least one of text information, image information, or voice information in response to the question items, and
the answer output unit outputs at least one of the text information, the image information, or the voice information input by the breeder to the medical terminal.

14. An animal medical care support server that supports medical care of an animal, the server comprising:
a medical inquiry storage unit that stores question items in association with animal species;
an animal species specifying unit that specifies the animal species of the animal scheduled to receive medical examination at a veterinary medical institution;
a question transmission unit that reads out the question items corresponding to the specified animal species and transmits them to a portable terminal operated by a breeder of the animal;
an answer reception unit that receives answers to the questions from the portable terminal; and
an answer output unit that outputs the answer to a medical terminal of a medical personnel of the veterinary medical institution.

15. A non-transitory computer-readable storage medium storing a program that supports medical care of an animal for causing a computer to execute:
a step of storing question items in association with animal species;
a step of specifying the animal species of the animal scheduled to receive medical examination at a veterinary institution;
a step of reading out the question items corresponding to the specified animal species and transmitting them to a portable terminal operated by a breeder of the animal;
a step of receiving answers to the question items from the portable terminal; and
a step of outputting the answer to the medical terminal of a medical personnel of the veterinary medical institution.
